(19) United States
(12) Patent Application Publication
(10) Pub. No.: US 2026/0165268 A1
(43) Pub. Date: Jun. 18, 2026

(54) METHODS OF CANNABACEAE PLANT BREEDING

(71) Applicant: Verda Bio LLC, Seattle, WA (US)

(72) Inventors: Jessica Tonani, Seattle, WA (US); Kersten Gaba, Seattle, WA (US)

(21) Appl. No.: 19/100,865

(22) PCT Filed: Aug. 7, 2023
(86) PCT No.: PCT/US2023/071765

§ 371 Date: Feb. 3, 2025

Related U.S. Application Data
(60) Provisional application No. 63/370,758, filed on Aug. 8, 2022.

(51) Int. Cl. A01H 1/02; A01H 1/04; A01H 3/04; A01H 5/02; A01H 6/28

(52) U.S. Cl. CPC A01H 1/021; A01H 1/04; A01H 3/04; A01H 5/02; A01H 6/28

(57) ABSTRACT

Alternative methods of Cannabaceae plant breeding are provided. More specifically, methods of breeding a quasi-dihaploid Cannabaceae plant are provided.

### CLAIM OF PRIORITY UNDER 35 U.S.C. § 119

The present Application for Patent claims benefit of Provisional Application 63/370,758 entitled “METHODS OF CANNABACEAE PLANT BREEDING” filed Aug. 8, 2022, and hereby expressly incorporated by reference herein.

### BACKGROUND

### Field

This invention generally relates to alternative methods of Cannabaceae plant breeding.

### Background

Plants of the family Cannabaceae possess commercial value and have many uses and applications which arise from the natural products that are extracted from their flowers, stalks, roots, and seeds. For instance, hops are extracted from the flowers of Humulus plants in this family. Hemp has multiple uses, including food and as a fiber for making clothing, rope, etc. Likewise, Cannabis plants have long been considered to have medicinal properties.

### BRIEF DESCRIPTION OF THE DRAWINGS

FIG. 1 is a graphical depiction of a typical prior art method of uniparental genome elimination (UGE).

FIG. 2 illustrates a method used in Example 1.

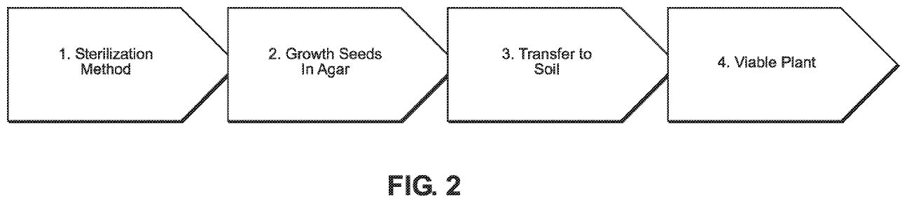

FIG. 3 shows a seed obtained with methods of the invention.

FIG. 4 is a graphical comparison of the prior art and the invention. In the left panel, which represents the prior art utilizing UGE, (1) a standard ploidy plant is used (in this example a diploid plant); (2) potential eggs (maternal); (3) eggs are pollinated with pollen from a related species “cousin pollen” (striped) and stress is induced to promote fast seed development (4) “cousin” DNA is recognized as foreign and eliminated; remaining haploid embryos are shown; (5) methods are used to duplicate haploid material and a stable dihaploid is obtained. In the right panel, which represents the invention, (1) a >2 ploidy plant is used; (2) potential eggs can range from haploid to multi-ploidy (maternal); (3) the eggs are pollinated with “cousin” pollen (striped) and stress is induced to induce fast seed development; (4) cousin DNA is recognized as foreign and eliminated and remaining embryos are shown; a subset of the embryos contain two sets of chromosomes from the maternal material and are stable quasi-dihaploids.

### SUMMARY

Some embodiments of the invention relate to a method of breeding a quasi-dihaploid Cannabaceae plant. The method can include one or more of the steps of: comprising: obtaining a flowering female polyploid Cannabaceae plant; pollinating the female polyploid Cannabaceae plant with a related species to obtain a pollinated Cannabaceae plant; inducing stress in the pollinated Cannabaceae plant; growing a seed of the pollinated Cannabaceae plant to maturity; and/or germinating the seed and selecting for a quasi-dihaploid Cannabaceae plant for breeding.

In some embodiments, the quasi-dihaploid Cannabaceae plant can have at least 50% homozygosity obtained in less than 7 generations of breeding.

In some embodiments, the quasi-dihaploid plant can have increased homozygosity compared to a previous generation that is sufficient to eliminate at least one breeding cycle.

In some embodiments, the obtaining step can include creating a Cannabaceae triploid by treating a Cannabaceae plant with one or more of: treatment of shoot tips, tissue culture samples, clippings, or other plant samples with colchicine and/or oryzalin and/or any compound capable of inhibiting or altering meiosis; and/or pre-treating germinating seeds with colcemid and/or colchicine and/or any compound capable of inhibiting or altering meiosis; and/or genomic modification of plants.

In some embodiments, the obtaining step can further include growing a treated Cannabaceae plant to maturity to obtain a matured plant prior to the pollinating step and testing the matured plant for sex by genetic methods and/or by inducing maturity and monitoring sex development.

In some embodiments, the obtaining step can include allowing flowering by light for photosensitive varieties or time for day-neutral varieties.

In some embodiments, the obtaining step can include allowing flowering by induction by a chemical. In some embodiments, the chemical can be a hormone.

In some embodiments, the Cannabaceae plant is Cannabis and the related plant is Humulus. In some embodiments, the Cannabaceae plant is Humulus and the related plant is Cannabis.

In some embodiments, the stress induction step is performed within 1 week of pollination.

In some embodiments, the stress induction is performed by one or more of environmental stress (e.g., heat, light or nutrient and/or watering modification); and/or hormonal induced stress induced by auxin, gibberellins, cytokinin, abscisic acid, ethylene, and/or the like; and/or chemical induced stress from a pesticide, silver nitrate, sodium thiosulfate, and/or the like.

In some embodiments, the seed germination step can include one or more of tissue culture, direct planting, and/or alternative methods.

In some embodiments, the flowering female polyploid is a flowering female triploid or greater ploidy.

In some embodiments, the selecting step can include selecting a plant with one or more of: resistance to a pathogen, resistance to a pest, tolerance to drought, tolerance to extreme temperatures, annual or perennial life cycles, and/or a desired cannabinoid and/or terpene profile.

In some embodiments, selection can include genomic and or analytical chemistry methods prior to maturity of the plant.

In some embodiments, selection can occur after maturity of the plant.

Some embodiments of the invention relate to a plant or plant part produced by any of the methods described herein.

### DETAILED DESCRIPTION

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which variations and additions do not depart from the present technology. Hence, the following description is intended to illustrate some particular embodiments of the technology, and not to exhaustively specify all permutations, combinations and variations thereof.

As used herein, the singular form “a,” “an” and “the” include plural referents unless the context clearly dictates otherwise.

The recitation herein of numerical ranges by endpoints is intended to include all numbers subsumed within that range (e.g., a recitation of 1 to 5 includes 1, 1.5, 2, 2.75, 3, 3.80, 4, 4.32, and 5).

Where the term “about” is used herein explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range, or within 1% of the given value or range.

The expression “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

As used herein, the term “Cannabis” refers to the genus of flowering plants in the family Cannabaceae regardless of species, subspecies, or subspecies variety classification. At present, there is no general consensus whether plants of genus Cannabis are comprised of a single or multiple species. For example some describe Cannabis plants as a single species, C. sativa L., with multiple subspecies while others classify Cannabis plants into multiple species, most commonly as C. sativa L. and C. indica Lam. and sometimes additionally as C. ruderalis Janisch., depending on multiple criteria including morphology, geographic origin, chemical content, and genetic measurements. Regardless, all plants of genus Cannabis can interbreed and produce fertile offspring. As used herein, the expression “Cannabis plant” includes hemp plant and as such, the expression “quasi-dihaploid Cannabaceae plant” includes triploid hemp plant. As used herein, the expression “Cannabis plant” includes high THC plants (often referred to in law as marijuana), the expression “quasi-dihaploid Cannabaceae plant” includes triploid high-THC plant.

As used herein, the term “cannabinoid” refers to a chemical compound belonging to a class of secondary compounds commonly found in plants of genus Cannabis, but also encompasses synthetic and semi-synthetic cannabinoids and any enantiomers thereof. In an embodiment, the cannabinoid is a compound found in a plant, e.g., a plant of genus Cannabis, and is sometimes referred to as a phytocannabinoid. In one embodiment, the cannabinoid is a compound found in a mammal, sometimes called an endocannabinoid. In one embodiment, the cannabinoid is made in a laboratory setting, sometimes called a synthetic cannabinoid. In one embodiment, the cannabinoid is derived or obtained from a natural source (e.g., a plant) but is subsequently modified or derivatized in one or more different ways in a laboratory setting, sometimes called a semi-synthetic cannabinoid.

As used herein, the expression “% w/w” or “% by weight” is calculated based on dry weight of the total material unless there is a specific and explicit other basis for calculation.

Dihaploid plant production can be carried out using multiple methods including anther cultures or pollen culture. One of the most common is using uniparental genome elimination (UGE), which is an innovative technique used to obtain haploid plants from the cross between two different parents. UGE leads to the elimination of one parental genome, resulting in a haploid plant with only the genome of the other parent.

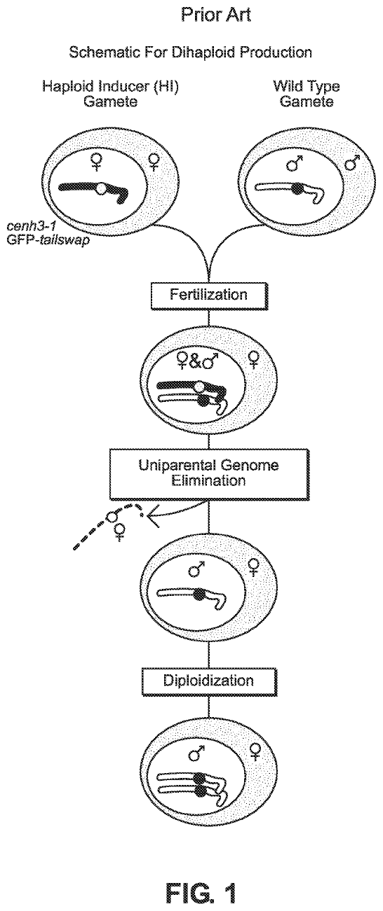

The basic steps involved in dihaploid plant production using uniparental genome elimination is depicted in FIG. 1 and described below:
Cross two different parents: Begin by crossing two distinct parental plants, which often are from two different species or varieties.
Induce genome elimination: After the cross, the resulting hybrid embryo contains genetic material from both parents (maternal and paternal genomes). The UGE process involves manipulating the conditions to favor the elimination of one of the parental genomes.
Haploid embryo formation: Through UGE, the embryo's genome is reduced to a haploid state, containing half the usual number of chromosomes.
Embryo rescue and chromosome doubling: Embryo rescue techniques involve transferring the haploid embryos to a suitable growth medium that provides the necessary nutrients and conditions for further development at this time chromosome doubling is also often done in order to create stable, homozygous lines.

This invention relates to the production of a quasi-dihaploid Cannabaceae plant. Dihaploid refers to a haploid cell in which its nucleus contains two copies of the same set of chromosomes. Dihaploid plants would have the same benefits of a highly inbred crop. A quasi-dihaploid plant, as used herein, refers to a plant that has at least 50, 55, 60, 65, 70, 75, 80, 85, 90 or more % homozygosity obtained in less than 7, 6, 5, 4, 3, 2, or 1 generations of breeding. In some embodiments, the quasi-dihaploid plant refers to a plant with an increase in homozygosity compared to a previous generation that is sufficient to eliminate at least one breeding cycle. In some embodiments, it is sufficient to eliminate at least 2, 3, 4, 5 or more breeding cycles. Variability can be due to crossing over events and the like. Thus, this invention provides methods for Cannabaceae plant breeding with limited variability in prodigy.

Percent homozygosity refers to the proportion of an individual's genome in which the two copies of a particular DNA sequence are identical. In other words, it measures the extent to which an individual has inherited identical genetic information between generations for a specific region (homozygous alleles). Percent identity as used herein refers to the comparison of the homozygous alleles between generations. Percent identity is determined by comparing a statistically significant number of the homozygous alleles of the two generations. For example, a percent identity of 90% between one generation and another generation means that the two generations have the same allele at 90% of their loci.

Some embodiments of the invention relate to a method of breeding a quasi-dihaploid Cannabaceae plant. The method can include one or more of the following steps:
1. obtaining a flowering polyploid female Cannabaceae plant with greater than a diploid genome;
2. pollinating the female polyploid Cannabaceae plant with a related species to obtain a pollinated Cannabaceae plant;
3. inducing stress in the pollinated Cannabaceae plant;
4. growing a seed of the pollinated Cannabaceae plant to maturity; and/or
5. germinating the seed and selecting for a quasi-dihaploid Cannabaceae plant for breeding.

As used herein, the term “diploid” refers to organisms or cells with two complete chromosome sets (2n), typically in the somatic cells of a plant. A Cannabis sativa diploid (2n) plant with a complete set of chromosomes has 20 chromosomes. Thus, a plant with greater than a diploid genome refers to a plant with more than two chromosome sets. As used herein, the term “polyploid” refers to a plant having more than the usual number (two) of chromosome sets, including but not limited to having three or more chromosome sets, four or more, five or more, etc.

A true polyploid will have the extra chromosome sets in all cells, but ploidy can vary between tissues and is sometimes not passed on to the seeds. Polyploid can refer to organisms with three or more complete chromosome sets in all somatic cells. Likewise, in some cases, as indicated by context, polyploid can also refer to organisms with three or more complete chromosome sets in one or more tissues. Polyploids can include, but are not limited to, triploids (3n), tetraploids (4n), hexaploids (6n), and octaploids (8n). As used herein, the expression “stable tetraploid plant” refers to a plant that retains its tetraploid number in some, most, or all tissues for a few months and/or through multiple generations.

The term “dihaploid line” refers to stable inbred lines created from breeding or genetic manipulation process that results in a plant containing only half the normal number of chromosomes in its cells. These haploid plants are then used in subsequent steps to create dihaploid lines. Such dihaploid lines can be created from several methods including spontaneous elimination of chromosomes in some species, but the most common methods include culturing of haploid tissue (often pollen or immature embryos) to develop plantlets containing 1n chromosomes. These plantlets can then have their chromosomes “doubled” using chemicals to produce plantlets with 2n chromosomes. The progeny of these plantlets are named “dihaploid” because their pairs of chromosomes are essentially identical. This results in homozygosity approaching 100% (fully homozygous). Accordingly, plants from fully homozygous dihaploid lines do not display segregating traits (i.e., they are stable).

“Flowering” or “flowering phase” (also called “bud cycle”) refers to the period in the plant life cycle during which the plant produces buds and flowers. This is the reproductive phase of plant growth. Cannabis is predominantly dioecious, having female and male reproduction parts on separate plants. Flowering is the gametophytic or reproductive state of Cannabis. In preferred commercial approaches to production of Cannabis flower, only female plants are selected for cultivation. For some cultivars, the switch from the vegetative stage to the flowering stage can be light-dependent. Some cultivars are autoflowering, meaning they switch to the flowering stage automatically, typically with age rather than being triggered by day length.

### Obtaining a Flowering Polyploid Female Cannabaceae Plant

In the obtaining step of the invention, a flowering polyploid female Cannabaceae plant is obtained. The plant can be a triploid, tetraploid, hexaploid, or the like. Plants of the family Cannabaceae, as used herein, refers to any member of the Cannabaceae family of plant organisms including, but not limited to, Celtis, Cannabis, and Humulus plants.

A polyploid can be obtained by any method known in the art, such as, but not limited to:
treatment of shoot tip, tissue culture sample, clipping and/or other plant sample with any compound that can inhibit or alter meiosis. Examples of such compounds can include, but are not limited to, colchicine, colcemid, oryzalin, and/or any compound which can inhibit or alter meiosis. Example protocols can be found in, for example, Bagheri, M., Mansouri, H. 2015. “Effect of Induced Polyploidy on Some Biochemical Parameters in Cannabis sativa L.” Applied Biochemistry and Biotechnology 175: 2366-2375. https://doi.org/10.1007/s12010-014-1435-8, which uses 0.1-2.0 w/v% colchicine for 24-48 hours and Parsons, J., Martin, S., James, T., Golenia, G., Boudko, E., Hepworth, S. 2019. “Polyploidization for the Genetic Improvement of Cannabis sativa.” Front Plant Sci. 10:476. https://doi:10.3389/fpls.2019.00476, which uses 20-150 uM oryzalin for 24-48 hours. Both of the foregoing references are fully incorporated by reference herein;
pre-treating germinating seeds with any compound that can inhibit or alter meiosis (including but not limited to colcemid, colchicine. and/or other compounds which can inhibit or alter meiosis). Example protocols can include, but are not limited to, using 0.1-0.3% colcemid (0-0.2% DMSO, 0-0.05% Tween 20) with seeds soaking 24-48 hours at 86° F. or about 86° F.; and/or
genomic modification of plants, which can modify standard meiosis and create plants with increased chromosomal numbers.

The treated or genetically modified plant can be then grown to maturity. On Cannabis plants, for example, the maturity can be determined by change in color of about half of the pistils (e.g., 40%, 45%, 50%, 55%, or 60%) from white to red and/or brown; change in color of about half of the trichomes (e.g., 40%, 45%, 50%, 55%, or 60%) from translucent to milky; and/or change in leaf color from green to yellow, with some leaves possibly curling, drying, and/or even falling from the plant. On Humulus plants, for example, burs will mature into cones and/or change in leaf color from green to yellow, with some leaves possibly curling, drying, and/or even falling from the plant.

A plant can be tested for sex by genetic methods, and/or by inducing maturity and monitoring sex development, and/or any method known in the art. A female plant can be then selected and used in the invention.

The mature plant can be induced into its flowering phase by light for photosensitive varieties, or by time for day-neutral varieties, and/or via induction by use of chemicals (including hormones). Hormones can include but are not limited to auxins, florigen, ethylene, and/or the like.

### Pollinating the Female Polyploid Plant With a Related Species

In the pollinating step of the invention, a related species can be used. The term “related species” can refer to related organisms that are genetically similar, but do not undergo interbreeding under normal circumstances. For example, a Cannabis plant can be pollinated by a Humulus plant. For example, a Humulus plant can be pollinated by a Cannabis plant.

In the pollinating step of the invention, a divergent variety can be used. The term “divergent variety” can refer to plants within the same species that do not normally interbreed. For example, Humulus Japonica can pollinate a Humulus Lupulus plant.

### Inducing Stress in the Pollinated Plant

In some embodiments, stress can be induced in the pollinated plant within 1 week of pollination. For example, it can be induced within 7, 6, 5, 4, 3, 2, 1 day, or a fraction of a single day of pollination. Examples of inducing stress can include, but are not limited to one or more of environmentally induced stress, hormonally induced stress, chemically induced stress, and/or the like.

### Environmentally Induced Stress

In some embodiments, the pollinated plant is subjected to environmentally induced stress which can include one or more of heat, light, nutrients/salt, watering modifications, and/or the like. In some embodiments, a plant can be subjected to heat treatment. Example heat treatment can include, but is not limited, to subjecting plants to at least 80° F. temperature for at least 24 hours. In some embodiments, the plants are subjected to heat for 70°, 80°, 90°, 100° F. or more for at least 12, 18, 24, 48 hours or more. In some embodiments, a plant can be subjected to light treatment using variable light cycles, including decreasing light brightness during “day-light” hours to induce scenarios that may mimic winter or turning lights off during “day-light” for, as an example, 1-hour periods. In some embodiments, a plant can be subjected to stress by treatment enabling NaCl accumulation per, for example, Yep B., Gale N. V., Zheng Y. 2020. “Aquaponic and Hydroponic Solutions Modulate NaCl-Induced Stress in Drug-Type Cannabis sativa L.” Front. Plant Sci. 11:1169. https://doi:10.3389/fpls.2020.01169, which uses a hydroponic solution of 1-40 mM NaCl. In some embodiments, a plant can be subjected to watering modifications of inducing drought scenarios by drastically decreasing watering similar to Jiang, Y., Sun, Y., Zheng, D., Han, C., Cao, K., Xu, L., Liu, S., Cao, Y., Feng, N. 2021. “Physiological and transcriptome analyses for assessing the effects of exogenous uniconazole on drought tolerance in hemp (Cannabis sativa L.)” Sci. Rep. 11:14476. https://doi.org/10.1038/s41598-021-93820-6. Both of the foregoing references are fully incorporated by reference herein.

### Hormonally Induced Stress

In some embodiments, the pollinated plant is subjected to hormone-induced stress by subjecting the plant to one or more hormones. The hormone can include, but is not limited to an auxin, gibberellin, cytokinin, abscisic acid, ethylene, and/or the like. For example, a plant can be stressed using 100 uM abscisic acid (ABA) treatment per the Jiang et al. protocol, cited above.

### Chemically Induced Stress

In some embodiments, the pollinated plant is subjected to one or more chemical stressors. The chemical stressors can include, but are not limited to, one or more of a pesticide, silver nitrate, sodium thiosulfate, or any other similar chemical. For example, a plant can be subjected to 2-Thiourasil at a level of 15-30 ug (e.g., 15, 16, 18, 20, 22, 24, 26, 28, or 30 ug) per protocol from Heslop-Harrison, J. 1960. “Suppressive Effects of 2-Thiouracil on Differentiation and Flowering in Cannabis sativa.” Science 132:1943-1944. https://doi.org/10.1126/science.132.3444.1943, which is hereby fully incorporated by reference herein.

In some embodiments, hormonally induced stress can include pollinating a plant with pollen from a related species. In specific embodiments, the plant can be in contact with the pollen either diluted (e.g., in a 1:10 mixture of pollen:lycopodium, pollen:flour) or non-diluted, completely covering the plant pistils. Pollen can be freshly collected or aged and can be placed on flowers via physical means (such as a brush) or via air (blown onto pistils) for a period of time. The period of time can be 1 hour to multiple days, for example, 1, 2, 5, 12, or 24 hours or 1, 2, 3, 4, 5, or more days. Once the period of time has passed, the plants can be sprayed with a hormone solution. In some embodiments, the hormone solution can be optionally mixed in formulations containing additional compounds including but not limited to dimethyl sulfoxide (DMSO), Tween®-20, and/or other substances that can increase plant uptake. Example solutions sprayed onto the plants include, but are not limited to gibberellic acid at a 0.1-2% (e.g., 0.1, 0.2, 0.4, 0.8, 1, 1.2, 1.4, 1.8, or 2%) in a Tween-20 solution and a 0.02-2% (e.g., 0.02, 0.04, 0.08, 1, 1.2, 1.4, 1.8, or 2%) 2-4 Dichlorophenoxyacetic Acid. Efficiency of the protocol can be seen when the plants undergo multiple pollination/treatment cycles over a period of hours to weeks (e.g., 1, 2, 3, 5, 24, 48, or more hours or 1, 2, 3, 4, or more weeks). In some embodiments, variety-specific optimization can include the treatment of plants with greater than one hormonal mixture between cycles (e.g., 1, 2, 3, 4, or more hormonal mixtures).

Without being bound by theory, the inducing of stress can cause the plant to induce replication or seed formation. During the development of the seed, there will come a point at which time the cells can recognize the foreign pollen as foreign and shed or lose the foreign chromosomes. In other words, the polyploid plant can recognize the foreign pollen enough to start seed development, but not enough to replicate the foreign DNA.

### Growing a Seed of the Pollinated Cannabaceae Plant to Maturity

In some embodiments, the plant can be allowed to grow to maturity using any method known in the art, such as, but not limited to soil, hydroponic methods, aeroponic methods, tissue culture, and/or alternative methods or accepted plant growth. Plants can be grown indoors, in greenhouses, or outdoors.

### Germinating the Seed and Selecting for a Quasi-Dihaploid Cannabaceae Plant

The germinating step can include any method known in the art, such as, but not limited to, tissue culture, direct planting, and/or alternative methods. A quasi-dihaploid Cannabaceae plant can be selected for by screening using genomic analysis methods such as cytology, DNA sequencing, and/or the like.

In some embodiments, the selecting step involves selecting a plant with a desirable phenotype such as, but not limited to, resistance to a pathogen, resistance to a pest, tolerance to drought, tolerance to extreme temperatures, annual or perennial life cycles, and/or a desired cannabinoid and/or terpene profile. Desired cannabinoid and/or terpene profiles are provided in the following section (“Quasi-Dihaploid Cannabaceae Plant”).

Selection can occur early utilizing genomic, analytical chemistry and/or other methods or can occur after plants are grown to maturity and selected based on final phenotype of a mature plant.

### Quasi-Dihaploid Cannabaceae Plant

Some embodiments of the invention relate to a plant or plant part produced by any of the methods disclosed herein.

In some embodiments, the present technology also relates to a quasi-dihaploid Cannabaceae plant comprising a resistance to specific pathogens including Hop Latent Viroid, Lettuce Chlorosis Virus, Cannabis Cryptic Virus, Tobacco Mosaic Virus, Beet Curly Top Virus, Golovinomyces, Botrytis, Fusarium, Pythium, and/or the like.

In some embodiments, the present technology also relates to a quasi-dihaploid Cannabaceae plant comprising a resistance to specific pests including mites, aphids, thrips, gnats, grasshoppers, leafhoppers, and/or the like.

In some embodiments, the present technology also relates to a quasi-dihaploid Cannabaceae plant comprising tolerance of drought or extreme temperatures.

In some embodiments, the present technology also relates to a quasi-dihaploid Cannabaceae plant comprising annual or perennial life cycles.

In some embodiments, the present technology also relates to a quasi-dihaploid Cannabaceae plant comprising a desired cannabinoid profile and/or terpene profiles as described in the following paragraphs. In some embodiments, the present technology also relates to a quasi-dihaploid Cannabaceae plant comprising mono-terpenes (i.e., terpenes that consist of two isoprene units and have the molecular formula C10H16) at levels of between about 0.01% and about 30% w/w, for example, 0.1% w/w, 1%, 5%. 10%, 15%, 20%, 25%, 30% or more w/w in dried flower. Examples include but are not limited to myrcene and limonene.

In some embodiments, the present technology also relates to a quasi-dihaploid Cannabaceae plant comprising sesquiterpenes (i.e., terpenes that are C15-terpenoids built from three isoprene units) at levels of between about 0.01% and about 30% w/w for example, 0.1% w/w, 1%, 5%, 10%, 15%, 20%, 25%, 30% or more w/w in dried flower. A non-limiting example is β-caryophyllene.

In some embodiments, the present technology also relates to a quasi-dihaploid Cannabaceae plant comprising a cannabidivarinic acid (CBDVA) content, in dried flower thereof, of between about 0.1% and about 30% w/w, or between about 0.1% and about 20% w/w, or between about 0.1% and about 10% w/w, or between about 1% and about 20% w/w, or between about 5.0% and about 20% w/w, or between about 5% and about 15% w/w. In some embodiments, the range can be between about 0.1% and about 30% w/w, for example, 0.1% w/w, 1%, 5%, 10%, 15%, 20%, 25%, 30% or more w/w CBDVA in dried flower.

In some embodiments, the present technology also relates to a quasi-dihaploid Cannabaceae plant comprising a cannabichromenic acid (CBCA) content, in dried flower thereof, of between about 0.1% and about 30% w/w, or between about 0.1% and about 20% w/w, or between about 0.1% and about 10% w/w, or between about 1% and about 20% w/w, or between about 5.0% and about 20% w/w, or between about 5% and about 15% w/w. In some embodiments, the range can be between about 0.1% and about 30% w/w, for example, 0.1% w/w, 1%, 5%, 10%, 15%, 20%, 25%, 30% or more w/w CBCA in dried flower.

In some embodiments, the present technology also relates to a quasi-dihaploid Cannabaceae plant comprising a tetrahydrocannabivarin (THCV) content, in dried flower thereof, of between about 0.1% and about 30% w/w, or between about 0.1% and about 20% w/w, or between about 0.1% and about 10% w/w, or between about 1% and about 20% w/w, or between about 5.0% and about 20% w/w, or between about 5% and about 15% w/w. In some embodiments, the range can be between about 0.1% and about 30% w/w, for example, 0.1% w/w, 1%, 5%, 10%, 15%, 20%, 25%, 30% or more w/w THCV in dried flower.

In some embodiments, the present technology also relates to a quasi-dihaploid Cannabaceae plant comprising a Δ⁸-tetrahydrocannabinol (Δ⁸-THC) content, in dried flower thereof, of between about 0.001% and about 0.01% w/w; or between about 0.002% and about 0.005% w/w, or between about 0.003% and about 0.005% w/w. In some embodiments, the range can be between about 0.1% and about 30% w/w, for example, 0.1% w/w, 1%, 5%, 10%, 15%, 20%, 25%, 30% or more w/w Δ⁸-THC in dried flower.

In some embodiments, the present technology also relates to a quasi-dihaploid Cannabaceae plant comprising a cannabigerolic acid (CBGA) content, in dried flower thereof, of between about 0.01% and about 20% w/w, or between about 0.05% and about 0.3% w/w, or between about 0.07% and about 0.3% w/w. In some embodiments, the range can be between about 0.1% and about 30% w/w, for example, 0.1% w/w, 1%, 5%, 10%, 15%, 20%, 25%, 30% or more w/w CBGA in dried flower.

In some embodiments, the present technology also relates to a quasi-dihaploid Cannabaceae plant comprising a cannabidiol (CBD) content, in dried flower thereof, of between about 0% and about 0.5% w/w, or between about 0% and about 0.3% w/w, or between about 0% and about 0.2% w/w. In some embodiments, the range can be between about 0.1% and about 30% w/w, for example, 0.1% w/w, 1%, 5%, 10%, 15%, 20%, 25%, 30% or more w/w CBD in dried flower.

In some embodiments, the present technology also relates to a quasi-dihaploid Cannabaceae plant comprising a cannabidiol acid (CBDA) content, in dried flower thereof, of between about 0% and about 20% w/w, or between about 0% and about 15% w/w, or between about 0% and about 10% w/w. In some embodiments, the range can be between about 0.1% and about 30% w/w, for example, 0.1% w/w, 1%, 5%, 10%, 15%, 20%, 25%, 30% or more w/w CBDA in dried flower.

In some embodiments, the present technology also relates to a quasi-dihaploid Cannabaceae plant comprising a Δ⁹-tetrahydrocannabinol (THC) content, in dried flower thereof, of between about 0.1% and about 1.0% w/w, or between about 0.1% and about 0.5% w/w, or between about 0.2% and about 0.5% w/w. In some embodiments, the range can be between about 0.1% and about 30% w/w, for example, 0.1% w/w, 1%, 5%, 10%, 15%, 20%, 25%, 30% or more w/w THC in dried flower.

In some embodiments, the present technology also relates to a quasi-dihaploid Cannabaceae plant comprising a Δ⁹-tetrahydrocannabinolic acid (THCA) content, in dried flower thereof, of between about 0.1% and about 30% w/w, or between about 0.1% and about 20% w/w, or between about 0.1% and about 10% w/w, or between about 1% and about 20% w/w, or between about 5.0% and about 20% w/w, or between about 5% and about 15% w/w. In some embodiments, the range can be between about 0.1% and about 30% w/w, for example, 0.1% w/w, 1%, 5%, 10%, 15%, 20%, 25%, 30% or more w/w THCA in dried flower.

In some implementations of these embodiments, the quasi-dihaploid Cannabaceae plant further comprises a α-pinene content, in dried flower thereof, of between about 0.04% and about 0.1% w/w, or between about 0.5% and about 0.1% w/w. In some embodiments, the range can be between about 0.1% and about 30% w/w, for example, 0.1% w/w, 1%, 5%, 10%, 15%, 20%, 25%, 30% or more w/w α-pinene in dried flower.

In some implementations of these embodiments, the quasi-dihaploid Cannabaceae plant further comprises a β-pinene content, in dried flower thereof, of between about 0.02% and about 0.5% w/w, or between about 0.3% and about 0.5% w/w. In some embodiments. the range can be between about 0.1% and about 30% w/w, for example, 0.1% w/w, 1%, 5%, 10%, 15%, 20%, 25%, 30% or more w/w β-pinene in dried flower.

In some implementations of these embodiments, the quasi-dihaploid Cannabaceae plant further comprises a myrcene content, in dried flower thereof, of between about 0.1% and about 0.5% w/w or between about % and about % w/w, or between about 0.2% and about 0.4% w/w. In some embodiments, the range can be between about 0.1% and about 30% w/w, for example, 0.1% w/w, 1%, 5%, 10%, 15%, 20%, 25%, 30% or more w/w myrcene in dried flower.

In some implementations of these embodiments, the quasi-dihaploid Cannabaceae plant further comprises a limonene content, in dried flower thereof, of between about 0.01% and about 0.05% w/w, or between about 0.2% and about 0.4% w/w. In some embodiments, the range can be between about 0.1% and about 30% w/w, for example, 0.1% w/w, 1%, 5%, 10%, 15%, 20%, 25%, 30% or more w/w limonene in dried flower.

In some implementations of these embodiments, the quasi-dihaploid Cannabaceae plant further comprises an ocimene content, in dried flower thereof, of between about 0% and about 0.005% w/w, or between about 0% and about 0.004% w/w. In some embodiments, the range can be between about 0.1% and about 30% w/w, for example, 0.1% w/w, 1%, 5%, 10%, 15%, 20%, 25%, 30% or more w/w ocimene in dried flower.

In some implementations of these embodiments, the quasi-dihaploid Cannabaceae plant further comprises a linalool content, in dried flower thereof, of between about 0.01% and about 0.03% w/w, or between about 0.1% and about 0.2% w/w. In some embodiments, the range can be between about 0.1% and about 30% w/w, for example, 0.1% w/w, 1%, 5%, 10%, 15%, 20%, 25%, 30% or more w/w linalool in dried flower.

In some implementations of these embodiments, the quasi-dihaploid Cannabaceae plant further comprises a γ-terpineol content, in dried flower thereof, of between about 0.02% and about 0.05% w/w, or between about 0.03% and about 0.05% w/w. In some embodiments, the range can be between about 0.1% and about 30% w/w, for example, 0.1% w/w, 1%, 5%, 10%, 15%, 20%, 25%, 30% or more w/w γ-terpineol in dried flower.

In some implementations of these embodiments, the quasi-dihaploid Cannabaceae plant further comprises a geraniol content, in dried flower thereof, of between about 0.001% and about 0.02% w/w, or between about 0.002% and about 0.01% w/w. In some embodiments, the range can be between about 0.1% and about 30% w/w, for example, 0.1% w/w, 1%, 5%, 10%, 15%, 20%, 25%, 30% or more w/w geraniol in dried flower.

In some implementations of these embodiments, the quasi-dihaploid Cannabaceae plant further comprises a β-caryophyllene content, in dried flower thereof, of between about 0.01% and about 0.05% w/w, or between about 0.02% and about 0.05% w/w. In some embodiments, the range can be between about 0.1% and about 30% w/w, for example, 0.1% w/w, 1%, 5%, 10%, 15%, 20%, 25%, 30% or more w/w β-caryophyllene in dried flower.

In some embodiments, the quasi-dihaploid Cannabaceae plant of the present technology comprises a total terpene content, in dried flower thereof, of between about 0.1% and about 2.0% w/w, or between about 0.1% and about 1.0% w/w, or between about 0.3% and about 0.8% w/w. In some embodiments, the range can be between about 0.1% and about 30% w/w, for example, 0.1% w/w, 1%, 5%, 10%, 15%, 20%, 25%, 30% or more w/w total terpene in dried flower.

### EXAMPLES

### Example 1

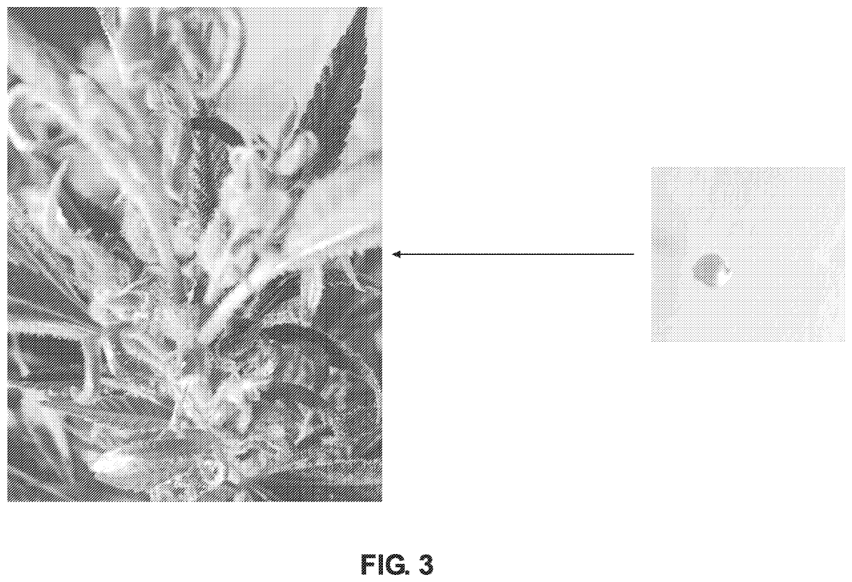

A female photoperiod-sensitive Cannabis plant seed was placed on an agar plate consisting of gelzan with 20% coconut water and 0.1% karomax for 48 hours. The sprouted seed was removed from the plate containing karomax and transplanted on to sterile agar (Murashige and Skoog medium with vitamins, gibberellic acid, sucrose, and gelzan). Once the plantlet reached 1.5 inches, it was carefully acclimated from the agar into a mixture of coco and soil under moisture domes. The moisture and ratio of coco to soil were slowly decreased over time. Immature plants were fully acclimated to be grown in soil under standard indoor growing conditions using this protocol. A polyploid, female plant created from the above protocol was selected and grown under standard indoor growing conditions in soil. Flower induction in the photosensitive plant was induced by a change in light cycle where the hours of daylight were decreased. Once mature pistils were visible, the Cannabis plant was pollinated with pollen obtained from a Humulus plant. The pollen was applied by hand and allowed to sit undisturbed on the flowers for 12 hours. After 12-hours, the plant flowers (including pistils) were sprayed with a solution containing gibberellic acid at a 0.1-2% in a Tween-20 solution. After 12 hours, the plant was washed with water, allowed to dry, and re-pollinated with Humulus pollen by hand. The plant was allowed to sit for 12 hours again, with pollen on the flowers (including pistils). After 12 hours, solution containing gibberellic acid at a 0.1-2% in a Tween-20 solution was sprayed on the flowers. After 12 hours, the plants were washed with water again and allowed to mature without further intervention outside of standard plant growth protocols (i.e., water, light, and nutrients). The plants were monitored for seed development. Image 1 shows the beginning of seed development and an immature seed (FIG. 5). Once the seeds reached a viable maturity level they were carefully collected, sterilized, and plated on agar plates (Murashige and Skoog medium with vitamins, gibberellic acid, sucrose and gelzan). FIG. 3 shows harvested seeds and plant pods from which the seed were harvested using this method. Plants grown from these seeds were confirmed to be quasi-dihaploid by methods such as cytology, DNA sequencing, and/or the like.

### Example 2

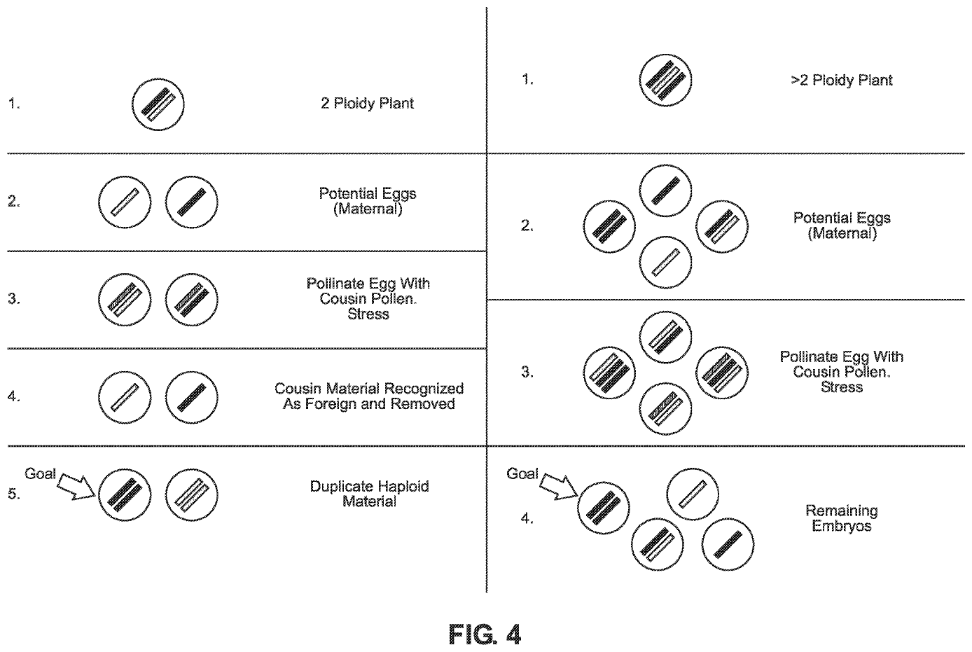

Variety-specific optimization using the protocol in Example 1 was performed. Modifications include timing or formulations of solutions applied to the plants. Acclimation protocols for plants transitioning from tissue culture to soil were also optimized. A female photoperiod-sensitive Cannabis plant seed was soaked in a solution of 0.1% colcemid and 0.1% DMSO for 48 hours (FIG. 4). The seed was removed from the solution and seed germination was induced by planting the seed in moist soil. The plantlet was grown in soil under standard indoor growing conditions using this protocol. Flower induction in the photosensitive plant was induced by a change in light cycle where the hours of daylight were decreased. Once mature pistils were visible, the Cannabis plant was pollinated with pollen derived from a Humulus plant. The pollen was applied by hand and allowed to sit undisturbed on the flowers for 12 hours. After 12-hours, the plant flowers (including pistils) were sprayed with a solution containing Gibberellic Acid at a 0.1-2% in a Tween-20 solution. After 12 hours, the plant was washed with water, allowed to dry, and re-pollinated with Humulus pollen by hand. The plant was allowed to sit for 12 hours again, with pollen on the flowers (including pistils). After 12 hours, 0.02-2% 2-4 dichlorophenoxyacetic acid was sprayed on the flowers. After 12 hours, the plants were washed with water again, re-pollinated and allowed to mature without further intervention outside of standard plant growth protocols (i.e., water, light, and nutrients). The plants were monitored for seed development. Once the seeds hit a viable maturity level they were carefully collected, sterilized, and plated on agar plates.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature, or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A method of breeding a quasi-dihaploid Cannabaceae plant comprising:
a. obtaining a flowering female polyploid Cannabaceae plant;
b. pollinating the female polyploid Cannabaceae plant with a related species to obtain a pollinated Cannabaceae plant;
c. inducing stress in the pollinated Cannabaceae plant;
d. growing a seed of the pollinated Cannabaceae plant to maturity; and
e. germinating the seed and selecting for a quasi-dihaploid Cannabaceae plant for breeding.

2. The method of claim 1, wherein the quasi-dihaploid Cannabaceae plant has at least 50% homozygosity obtained in less than 7 generations of breeding.

3. The method of claim 1, wherein the quasi-dihaploid plant has increased homozygosity compared to a previous generation that is sufficient to eliminate at least one breeding cycle.

4. The method of claim 1, wherein the obtaining step comprises creating a Cannabaceae triploid by treating a Cannabaceae plant with one or more of: treatment of shoot tips, tissue culture samples, clippings, or other plant samples with colchicine and/or oryzalin and/or any compound capable of inhibiting or altering meiosis; and/or pre-treating germinating seeds with colcemid and/or colchicine and/or any compound capable of inhibiting or altering meiosis; and/or genomic modification of plants.

5. The method of claim 1, wherein the obtaining step further comprises growing a treated Cannabaceae plant to maturity to obtain a matured plant prior to step (b) and testing the matured plant for sex by genetic methods and/or by inducing maturity and monitoring sex development.

6. The method of claim 1, wherein the obtaining step comprises allowing flowering by light for photosensitive varieties or time for day-neutral varieties.

7. The method of claim 1, wherein the obtaining step comprises allowing flowering by induction by a chemical.

8. The method of claim 6, wherein the chemical is a hormone.

9. The method of claim 1, wherein the Cannabaceae plant is Cannabis and the related plant is Humulus.

10. The method of claim 1, wherein the Cannabaceae plant is Humulus and the related plant is Cannabis.

11. The method of claim 1, wherein the stress induction step is performed within 1 week of pollination.

12. The method of claim 1, wherein the stress induction is performed by one or more of environmental stress (e.g., heat, light or nutrient and/or watering modification); and/or hormonal induced stress induced by auxin, gibberellins, cytokinin, abscisic acid, ethylene, and/or the like; and/or chemical induced stress from a pesticide, silver nitrate, sodium thiosulfate, and/or the like.

13. The method of claim 1, wherein the seed germination step comprises one or more of tissue culture, direct planting, and/or alternative methods.

14. The method of claim 1, wherein the flowering female polyploid is a flowering female triploid or greater ploidy.

15. The method of claim 1, wherein the selecting step comprises selecting a plant with one or more of: resistance to a pathogen, resistance to a pest, tolerance to drought, tolerance to extreme temperatures, annual or perennial life cycles, and/or a desired cannabinoid and/or terpene profile.

16. The method of claim 1, wherein selection comprises genomic and or analytical chemistry methods prior to maturity of the plant.

17. The method of claim 1, wherein selection occurs after maturity of the plant.

18. A plant or plant part produced by the method of claim 1.
